(19) United States
(12) Patent Application Publication
(10) Pub. No.: US 2025/0123293 A1
(43) Pub. Date: Apr. 17, 2025

(54) METHOD FOR EARLY DIAGNOSIS OF ACUTE MYOCARDIAL INFARCTION

(71) Applicant: The First Affiliated Hospital of Ningbo University, Ningbo City (CN)

(72) Inventors: Hanbin CUI, Ningbo City (CN); Jiajun YING, Ningbo City (CN); Junsong LIU, Ningbo City (CN); Jinsong CHENG, Ningbo City (CN); Ning HUANGFU, Ningbo City (CN); Hengyi MAO, Ningbo City (CN); Zhenwei LI, Ningbo City (CN)

(21) Appl. No.: 18/524,657

(22) Filed: Nov. 30, 2023

(30) Foreign Application Priority Data
Oct. 11, 2023 (CN) 202311309393.X

(51) Int. Cl. G01N 33/68

(52) U.S. Cl. CPC G01N 33/6893; G01N 2800/324; G01N 2800/50

(57) ABSTRACT

The present invention provides a method for early diagnosis of acute myocardial infarction and for predicting whether an individual is suffering from acute myocardial infarction. The method includes: providing a liquid sample derived from the individual; detecting a concentration or content of the biomarker in the liquid sample; and determining whether the individual is suffering from acute myocardial infarction according to the content. This method can be utilized for efficient detection and risk stratification of early myocardial infarction, accurate diagnosis and evaluation of patients with acute myocardial infarction and potential high-risk people, so as to improve the early diagnosis rate of acute myocardial infarction, guide the implementation of precise medical strategy, and thus improve the rescue level of acute myocardial infarction.

### CROSS-REFERENCE TO RELATED APPLICATIONS

The present application claims the priority of Chinese earlier application No. 202311309393.X filed on Oct. 11, 2023, all the content of which is incorporated by reference as a part of the present application.

### BACKGROUND OF THE INVENTION

### Field of the Invention

The present invention relates to the field of early diagnosis of myocardial infarction, and in particular to a biomarker combination, kit, system and use for diagnosis and risk stratification of a hyperacute phase of acute myocardial infarction (<6 hours).

### Description of the Related Art

Acute myocardial infarction (AMI) is myocardial necrosis caused by acute and persistent ischemia and hypoxia of coronary arteries. For diagnosis of AMI, the criteria of World Health Organization (WHO) have been followed always. If two of three indicators including typical chest pain, electrocardiogram changes and abnormal cardiac enzymes are met, the diagnosis can be made. With the discovery and understanding of new myocardial injury markers, the current diagnosis of AMI is mainly based on the criteria in the fourth edition of the “Universal Definition of Myocardial Infarction”, that is, cardiac troponin (cTn) is elevated and is higher than an upper limit of a normal value (at 99 percentile value of an upper limit of a reference value) at least once), and meanwhile there are clinical evidences of acute myocardial ischemia, including: (1) symptoms of acute myocardial ischemia; (2) new ischemic electrocardiogramanges; (3) newly emitted pathological Q waves; (4) imageological evidence of new loss of viable myocardium or regional wall motion abnormality; and (5) coronary thrombosis confirmed by coronary angiography or intraluminal imaging examination or autopsy. If the “5+1” criteria are met, AMI can be diagnosed.

The Clinical manifestation symptoms of AMI are often atypical. it is reported that among AMI patients in China, asymptomatic persons account for about 25%, and persons having atypical symptoms account for about 30%. Additionally, the accuracy of diagnosing AMI based on electrocardiogram alone is only about 60%. ST segment elevation with diagnostic significance is often manifested atypical in many cases, and pathological Q waves often appear 6-8 hours after onset. However, current electrocardiogram lacks specificity for the diagnosis of non-ST-elevation AMI (NSTEMI), so that the electrocardiogram diagnosis rate of early AMI is very low. However, 1-6 hours after the onset of early AMI is the prime time for thrombolytic therapy and interventional surgery, so that rapid diagnosis of early myocardial infarction within 6 hours of onset is a key step in determining treatment (J Am CollCardiol2021 November 30; 78 (22): 2218-2261, 2021 AHA/ACC/ASE/CHEST/SAEM/SCCT/SCMR Guideline for the Evaluation and Diagnosis of Chest Pain: Executive Summary: A Report of the American College of Cardiology/American Heart Association Joint Committee on Clinical Practice Guidelines).

It has shown by research in recent years that the detection of biochemical markers of myocardial injury plays an important role in the diagnosis, monitoring, risk assessment, prognosis and guidance of treatment of acute myocardial infarction. Currently, the most commonly used myocardial injury markers include creatine kinase isoenzyme (CK-MB), troponin (cTnI or CTNI), and myoglobin. As a marker of early heart injury, myoglobin lacks specificity for myocardium. CK-MB is fast and cost-effective, but still lacks specificity. Troponin has the highest accuracy, but its diagnostic performance is still significantly insufficient for patients in the hyperacute phase of onset (<6 hours), especially for patients with atypical symptoms and electrocardiograms.

AMI has the characteristics of high morbidity and high mortality, etc. The risk of sudden death is highest within a few hours after the onset. Meanwhile, the few hours after the onset is also a golden window period for medical rescue. Therefore, it is of great clinical value and social significance to find and establish a diagnostic marker of the hyperacute phase.

### BRIEF SUMMARY OF THE INVENTION

In view of the problems existed in the prior art, the present invention provides a method for diagnosing a hyperacute phase of acute myocardial infarction by analyzing a metabolite with significant differences in plasma samples of a patient with myocardial infarction in a hyperacute phase and of normal people, and screening out a series of new biomarkers that can conduct early diagnose of a risk of myocardial infarction, and establish a diagnostic model for early myocardial infarction, which is used for efficient detection of early myocardial infarction and assisting in clinically conducting risk stratification and clinical phenotype refinement of patients with acute myocardial infarction, thereby reducing erroneous diagnosis and improving a rescue level and a rescue effect.

The patient with “myocardial infarction in the hyperacute phase” described in the present invention are a group of population with similar characteristics, including: the hyperacute phase of acute myocardial infarction (within 6 hours after onset), acute chest pain, an acute coronary syndrome and the like disorders. According to the method provided by the present invention, it is mainly used for timely diagnosis of a patients who is considered to be with acute myocardial infarction (in the hyperacute phase), and meanwhile for identifying cardiogenic and non-cardiogenic causes of a patient with acute chest pain, so as to start a precision medical strategy as early as possible.

In one aspect, the present invention provides a method for diagnosing whether an individual has early myocardial infarction comprises detecting a concentration or an amount of a biomarker in liquid sample obtained from the individual. The marker is one or more selected from: pentadecanoate (15:0), tryptophan, laurate (12:0), methionine sulfoxide, (14 or 15)-methylpalmitate (a17:0 or i17:0) (methyl palmitate), acetylcarnitine (C2), 3-methyl-2-oxobutyrate, oxindolylalanine, asparagine, methionine, N-palmitoylglycine, carnitine, and phenylalanine. In an detail embodiments, the biomarker comprises a combination of above 13 makers.

In the present invention, through non-targeted metabolomics research, plasma samples from patients when they are admitted to hospital are collected, and the patients are divided into a diseased group and a non-diseased group according to whether the onset of their symptoms, such as chest tightness and chest pain, etc. are continuously not relieved, or recurred, and whether the symptoms are progressed to myocardial infarction in a short period (<6 hours) (the diseased group is a population that has progressed to myocardial infarction in a short period (<6 hours), and the non-diseased group is a population that has not progressed to myocardial infarction in a short period (<6 hours)), where the plasma troponin of the diseased group has not yet been increased. The plasma samples of the diseased group and the non-diseased group are analyzed by ultra performance liquid chromatography-tandem mass spectrometry (UPLC-MS/MS).

Statistical analysis (random forest, partial least squares-discriminant analysis (PLS-DA), LASSO regression analysis) and pathway enrichment analysis are used for determining the discovery of concentrated candidate biomarkers and determining the priority thereof, and screening out significantly different metabolites, and finally 13 biomarkers are obtained, which can be used for early diagnosis of the probability of acute myocardial infarction.

In some embodiments, a reagent for the biomarker for diagnosing whether early myocardial infarction occurs is a detection reagent prepared with the biomarker as a detection target, e.g., a sample pretreatment reagent, an antigen or an antibody and the like biological reagents and kits suitable for detection of the biomarker; and it can also be developed into a standardized reagent or kit suitable for LC-UV or LC-MS detection of the biomarker.

The biomarker includes a combination of pentadecanoate, tryptophan, laurate, methionine sulfoxide, methylpalmitate, acetylcarnitine, 3-methyl-2-oxobutyrate, oxindolylalanine, asparagine, methionine, N-palmitoylglycine, carnitine, and phenylalanine.

By investigating the concentration difference, AUC value ranking and significance ranking of the biomarkers in plasma samples between the diseased group and the non-diseased group at admission to hospital, 13 biomarkers are further selected from the aforementioned series of biomarkers that can significantly distinguish the diseased group from the non-diseased group. The biomarkers can be used for predicting the risk of early myocardial infarction more effectively, or can be used for establishing a diagnostic model to predict the risk of early myocardial infarction.

Further, the acute myocardial infarction includes a hyperacute phase, and the hyperacute phase refers to a time window range of the patient after the acute myocardial infarction occurs.

The time window range described in the present invention refers to the most effective golden time for thrombolytic therapy and interventional surgery after acute myocardial infarction, and is also a key link for effective treatment of early myocardial infarction.

Further, the acute myocardial infarction includes a hyperacute phase, and the hyperacute phase means that the patient will progress to acute myocardial infarction within 6 hours or less.

Further, the reagent is used for detecting a biomarker in a body fluid sample, including any one of blood, urine, saliva, and sweat.

Further, the reagent is used for detecting the presence or absence or relative abundance or concentration of a biomarker in a body fluid sample, or the degree or amplitude of changes in the content or quantity of a marker.

The presence or absence or content of the marker here is a relative concept. For example, compared with the non-diseased group, in the diseased group, the content of these specific markers is compared based on the diseased group or the non-diseased group as a benchmark. It may be that the content of certain markers in the diseased group is higher than that in the non-diseased group. This high level has a statistical difference, such as a significant or extremely significant increase. Therefore, when judgment is made about these markers, if a maker is a single marker, and if the content of the marker changes when the probability of a certain risk is increased, the change here may be a relative increase or a relative decrease. The difference in this relative increase or relative decrease has a significant difference, and of course, it may also be an extremely significant difference. Therefore, no matter what means is used for detection, a predetermined value can be used as a standard (cut-off value). Being higher than this value is considered as that the content has changed. Having such a result can be used as a prediction or diagnostic value.

Therefore, in some aspects, the markers described in the present invention can be obtained by detecting the content of the markers in a sample by any currently known method, such as liquid chromatography, gas chromatography, mass spectrometry, LC-MS, GC-MS, CC-MS, LC-MS-MS, NMR, immunochromatography test strip, immunoreaction chip, capillary electrophoresis, infrared spectroscopy, etc. The method can be used for early diagnosis of myocardial infarction as long as it can be used for detecting the content of the markers in the sample. As long as the content in the sample can be detected, it can be used for predicting or diagnosing the probability of a certain disease. It can be understood that the detection here is to detect an individual sample and then compare the detection result with a preset standard. The comparison result is used for judging or predicting the occurrence status of a disease. For example, it can be used for predicting the probability of early myocardial infarction. Such prediction or diagnosis is each whether it occurs within a certain period of time. Of course, such detection can be continuous detection, and the progression of the disease can be inferred as the content of certain substances changes.

In some embodiments, the relative abundance is a peak area of the biomarker in a detection spectrum obtained by high performance liquid chromatography-tandem mass spectrometry. For example, an average peak area of a certain biomarker measured in a control sample is 500, and an average peak area measured in a sample from a short-term non-surviving group of patients with myocardial infarction is 3,000, then the biomarker in the sample is considered to have abundance which is 6 times that in the control sample.

In some embodiments, any one or any combination of the 13 markers of the present invention can achieve the diagnosis of early myocardial infarction. Of course, these markers can also be used in conjunction with traditional biochemical indicators for joint detection. For example, the traditional biological indicators includecreatine kinase isoenzyme (CK-MB), troponin (cTnI or CTNI), and myoglobin.

In another aspect, the present invention provides a kit for early diagnosis of acute myocardial infarction, including a detection reagent for the biomarker as described above.

Further, the detection reagent for the biomarker includes a standard of the biomarker.

In a further aspect, the present invention provides a biomarker combination for early diagnosis of acute myocardial infarction, including a combination of any two or more of: pentadecanoate, tryptophan, laurate, methionine sulfoxide, methylpalmitate, acetylcarnitine, 3-methyl-2-oxobutyrate, oxindolylalanine, asparagine, methionine, N-palmitoylglycine, carnitine, and phenylalanine.

In still a further aspect, the present invention provides a diagnosis system for early diagnosis of acute myocardial infarction. The system includes a data analysis module for analyzing a predictive value of a biomarker, and the biomarker includes one or more of: pentadecanoate, tryptophan, laurate, methionine sulfoxide, methylpalmitate, acetylcarnitine, 3-methyl-2-oxobutyrate, oxindolylalanine, asparagine, methionine, N-palmitoylglycine, carnitine, and phenylalanine.

Further, the biomarker includes a combination of pentadecanoate, tryptophan, laurate, methionine sulfoxide, methylpalmitate, acetylcarnitine, 3-methyl-2-oxobutyrate, oxindolylalanine, asparagine, methionine, N-palmitoylglycine, carnitine, and phenylalanine.

Further, the data analysis module determines whether it is early myocardial infarction by substituting a detection value of a biomarker into a regression equation to calculate a predictive value for early diagnosis of acute myocardial infarction.

Further, the regression equation is a logistic regression equation which is obtained by constructing and training from the detection values of a biomarker of a known sample.

The detected marker as described in the present invention can be serum or plasma samples collected from patients with early myocardial infarction under any circumstances, i.e., serum or plasma samples collected from patients with early myocardial infarction at different times, in different regions, in different quantities, in different sexes, in different ages, and of the like conditions.

Further, the data analysis module determines whether it is early myocardial infarction by substituting a detection value of a biomarker into the logistic regression equation to calculate a predictive value for early diagnosis of acute myocardial infarction, thereby determining whether the patient will progress to acute myocardial infarction within 6-8 hours.

Further, the logistic regression equation is:

$$log ( \frac{p}{1 - p} ) = - 1.27416652 {9}^{*} + pentadecanoate + 1.078442206 * tryptophan - 1.27535689 * laurate + 1.32331323 * methionine sulfoxide - 1.010616893 * methylpalmitate - 1.450150532 * acetylcarnitine - 0.720980628 * 3 - methyl - 2 - oxobutyrate + 0.732863088 * oxindolylalanine + 0.494666685 * asparigine + 1.114785457 * methionine - 0.989479547 * N - palmitoylglycine + 2.207517356 * carnitine + 1.378071365 * phenylalanine ;$$

where p represents a critical threshold of the hyperacute phase of acute myocardial infarction.

Further, the risk of the patient being in the hyperacute phase of acute myocardial infarction is predicted to be high when p is greater than 0.53; and the risk of the patient being in the hyperacute phase of acute myocardial infarction is predicted to be low when p is less than 0.53.

Further, the system further includes a data storage module, a data input interface and a data output interface. The data storage module is used for storing a detection value of a biomarker. The data input interface is used for inputting the detection value of the biomarker. The data output interface is used for outputting a prediction result.

In yet still a further aspect, the present invention provides a method for early diagnosis of acute myocardial infarction, which is used for early diagnosis of acute myocardial infarction by analyzing a detection value of a biomarker. The biomarkers include one or more of: pentadecanoate, tryptophan, laurate, methionine sulfoxide, methylpalmitate, acetylcarnitine, 3-methyl-2-oxobutyrate, oxindolylalanine, asparagine, methionine, N-palmitoylglycine, carnitine, and phenylalanine.

Further, the reagent is used for detecting a biomarker in a body fluid sample, including any one of blood, urine, saliva, and sweat.

Further, the reagent is used for detecting the presence or absence or relative abundance or concentration of a biomarker in a body fluid sample, or the degree or amplitude of changes in the content or quantity of a marker.

Further, the method determines whether it is early myocardial infarction by substituting a detection value of a biomarker into a regression equation to calculate a predictive value for early diagnosis of acute myocardial infarction.

Further, the regression equation is a logistic regression equation which is obtained by constructing and training from the detection values of a biomarker of a known sample.

Further, the training is performed by constructing a training set, and training the training set with a training method to obtain a logistic regression equation; and the training set is a plasma sample of early myocardial infarction.

Further, the data analysis module determines whether it is early myocardial infarction by substituting a detection value of a biomarker into a logistic regression equation to calculate a predictive value for early diagnosis of acute myocardial infarction.

The method for screening out a patient with early myocardial infarction as provided by the present invention has the following beneficial effects:
1. 13 brand new biomarkers that can early predict the risk of myocardial infarction are screened out;
2. using this series of biomarkers and logistic regression analysis can effectively predict whether an individual has myocardial infarction;
3. it can realize early sensitivity and specificity diagnosis within 6 hours of onset;
4. the method is convenient and rapid, and the detection results are highly consistent with clinical gold standard test results; and
5. this method accurately locates the high-risk population of patients with acute myocardial infarction, and accurately evaluates the cardiovascular risk of this population, so as to facilitate early discovery and early intervention, promote early discovery and early treatment of myocardial infarction, and meet the urgent clinical needs.

### DETAILED DESCRIPTION

### (1) Diagnosis or Detection

Here diagnosis or detection is predicted to refer to the detection or assay of a biomarker in a sample, or the content of a biomarker of interest, such as absolute content or relative content, and then whether the individual from which the sample is provided may have or suffer from a certain disease or have the possibility of a certain disease is illustrated through the presence or absence or quantity of the biomarker of interest. The meanings of diagnosis and detection here are interchangeable. The result of this detection or diagnosis cannot be directly regarded as a direct result of suffering from a disease, but an intermediate result. If a direct result is obtained, it is necessary to confirm suffering from a disease through other auxiliary means such as pathology or anatomy. For example, the present invention provides a variety of new biomarkers that are associated with the occurrence of early myocardial infarction, and changes in the content of these markers are directly related to whether the patient suffers from early myocardial infarction.

(2) Association of a Marker or Biomarker with Early Myocardial Infarction

The marker and biomarker have the same meaning in the present invention. The association here means that the presence or change in the content of a certain biomarker in a sample is directly related to a specific disease or the progress of the disease. For example, a relative increase or decrease in the content indicates the possibility of suffering from the disease is higher than that of healthy people, or indicates the progress of the disease develops more seriously or develops from one stage to another. For example, a single marker or a combination of marker substances of multiple new markers of the present invention can be used for predicting whether early myocardial infarction will occur.

If a number of different markers in the sample appear at the same time or the content changes relatively, it means that the possibility of suffering from this disease is higher than that of healthy people. That is, among the types of markers, some markers have strong association with suffering from a disease, some markers have weak association with suffering from a disease, or some even have no association with a specific disease. One or more of the markers with strong association can be used as markers for diagnosing a disease, and those markers with weak association can be combined with strong markers to diagnose a certain disease to increase the accuracy of detection results. The disease here can be the process or progress of the disease, for example, developed from a better stage of a disease to a more malignant or serious stage, or even finally death.

For the numerous biomarkers found in the serum of the present invention, these markers all can be used for determining whether the patient is a patient with early myocardial infarction; and they can also be used for diagnosing or predicting the probability or possibility of early myocardial infarction. The markers here can be used as individual markers for direct detection or diagnosis. Selecting such a marker indicates that the relative change in the content of the marker is strongly associated with a patient with early myocardial infarction. Of course, it can be understood that simultaneous detection of one or more markers for early diagnosis of acute myocardial infarction can be selected. The normal understanding is that in some embodiments, selecting a biomarker with strong association for detection or diagnosis can achieve a certain standard of accuracy, for example an accuracy of 60%, 65%, 70%, 80%, 85%, 90% or 95%. Then it can be explained that these markers can obtain an intermediate value for diagnosing a certain disease, but it does not mean that it can directly confirm suffering from a certain disease.

Of course, you can also choose a differential marker with a larger AUC value as a diagnostic marker. The so-called strong and weak are generally calculated and confirmed through some algorithms, for example the contribution rate or weight analysis of markers and the probability of early myocardial infarction. Such a calculation method can be significance analysis (a p value or FDR value) and fold change, and multivariate statistical analysis mainly including principal component analysis (PCA) and partial least squares-discriminant analysis (PLS-DA).

(3) Patients with Early Myocardial Infarction

The patients with early myocardial infarction refers to patients who may progress to acute myocardial necrosis due to persistent myocardial ischemia, but the onset time is short (less than 6 hours) and the clinical phenotype is atypical, and the currently commonly-used troponin detection cannot clearly diagnose the patients. These patients may progress to large-area myocardial infarction, or the infarct area may be relatively limited with timely treatment, i.e., small-scale AMI.

Therefore, the prediction method provided by the present invention can quickly identify patients with early myocardial infarction among patients with myocardial injury or unstable angina pectoris and small-scale AMI, so as to provide early intervention and treatment.

(4) Epidemiological features of acute myocardial infarction: patients with symptoms that may radiate to the left upper limb, such as acute chest pain, chest tightness or throat tightness, including patients with new chest pain or acute exacerbation of existing chest pain. Such patients are accompanied or not accompanied by clinical symptoms such as profuse sweating, fever, tachycardias, fatigue, dizziness, syncope, hypotension and shock.

Electrocardiogram: ST segment (elevation or depression) and T wave (flattening or inversion) changes, where dynamic changes in the ST segment (elevation or depression ≥0.1 mV) are characteristic manifestations of coronary artery lesions. There are also some patients whose electrocardiogram has no changes or has changes lacking specificity.

Laboratory tests: myocardial injury markers such as troponin, creatine kinase, myoglobin, etc. have not yet been increased with diagnostic significance; and they may be accompanied by an increase in white blood cells, an increase in C-reactive protein, an increase in the erythrocyte sedimentation rate, etc.

### BRIEF DESCRIPTION OF THE DRAWINGS

FIG. 1 is a flow chart for screening biomarkers through 3 statistical methods: random forest, PLS-DA, and LASSO regression analysis in Example 1, where (a) is a schematic diagram of the results of screening biomarkers using the 3 statistical methods; (b) is a schematic diagram of analysis for the results of screening biomarkers using the 3 statistical methods respectively; and (c) is a schematic diagram of analysis for the results of screening biomarkers using the 3 statistical methods.

FIG. 2 is a ROC analysis chart of an individual biomarker asparagine in Example 2; and

FIG. 3 is a ROC analysis chart of a diagnostic model of early myocardial infarction constructed using 13 biomarkers in Example 2.

### DETAILED DESCRIPTION OF THE INVENTION

The present invention will be further described in detail with reference to accompanying drawings and examples, and it should be pointed out that the following examples are intended to facilitate the understanding of the present invention, without any limitation to it. The reagents used in the present examples are all known products and obtained by purchasing commercially available products.

### Example 1 Preliminary Screening of Biomarkers by Utilizing Metabolomics

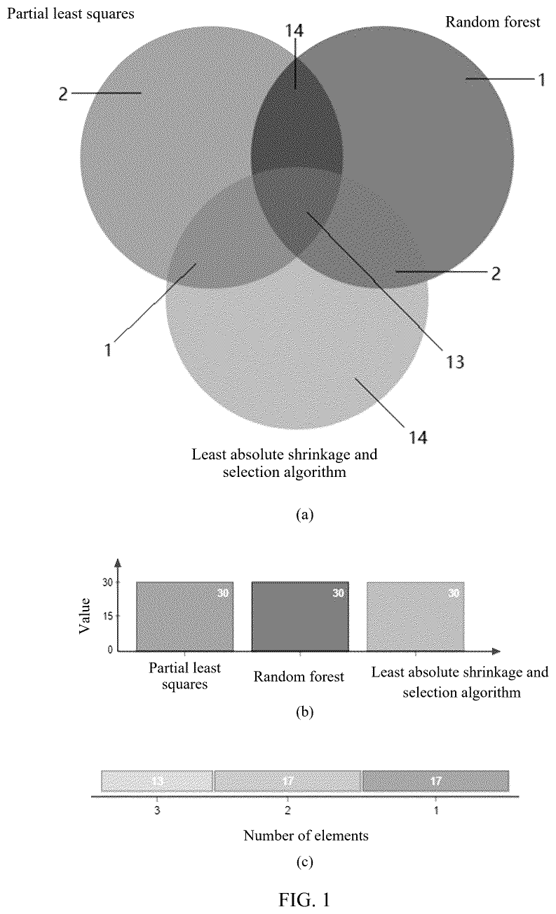

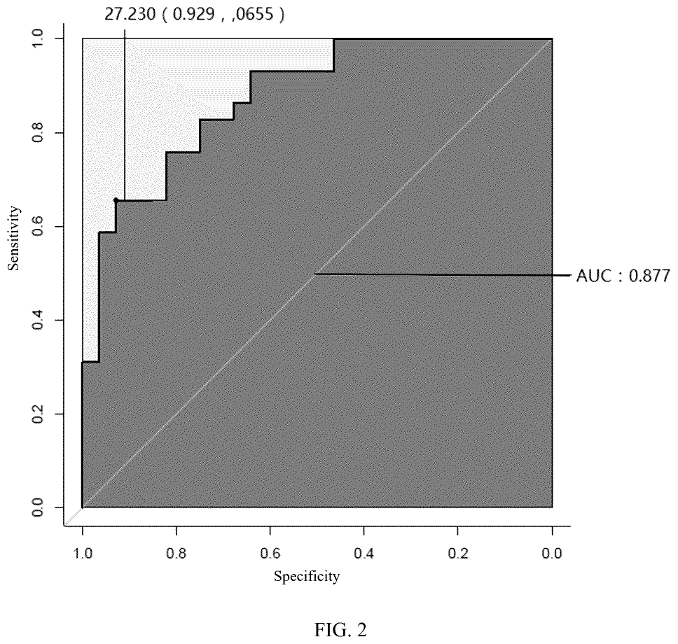

In this example, a non-targeted metabolomics study was first conducted to collect plasma samples from patients upon admission, and the patients were divided into a diseased group and a non-diseased group according to whether they progressed to early myocardial infarction within a short period of time after the onset. The serum samples of the diseased group and the non-diseased group were analyzed by ultra performance liquid chromatography-tandem mass spectrometry (UPLC-MS/MS). Statistical analysis (random forest, PLS-DA, LASSO regression analysis) and pathway enrichment analysis were used for determining the discovery of concentrated candidate biomarkers and determining the priority thereof, and screening out significantly different metabolites, and finally 13 biomarkers were obtained, which could be used for early diagnosis of the probability of acute myocardial infarction, which could be used for efficiently predicting the risk of early myocardial infarction, as shown in FIGS. 1 and 2.

Specific steps were as follows:

### 1. Experimental Method

### (1) Sample Collection

In this example, 200 samples of suspected acute myocardial infarction cases were collected, of which 86 cases developed into early myocardial infarction and 114 cases had no myocardial infarction. Patients with early acute myocardial infarction were individuals with acute myocardial infarction confirmed by coronary angiography and subsequent detection of a myocardial marker.

Baseline plasma samples upon admission from a discovery set were analyzed using untargeted metabolomics by high-performance liquid chromatography-tandem mass spectrometry for the aforementioned specimens. A total of 764 metabolites with known structures were identified on a non-targeted metabonomics platform, covering a wide range of biochemical pathways, including amino acids, lipids, carbohydrates, nucleotides, exogenous substances and intestinal microbial metabolism. After the metabolites detected in only less than 20% of the samples were removed, the remaining 528 metabolites were analyzed continually. We had observed that acute myocardial infarction had a profound impact on the serum metabolome, with 197 of 528 metabolites (75.2%) showing significantly changed levels (FDR<0.05) between a group that developed into myocardial infarction and a group that did not develop into myocardial infarction.

m/z ions were extracted from the original mass spectrometry data detected by LC-MS/MS, and the metabolites were identified by searching a database. A peak area was obtained by checking the chromatographic peak integration of a metabolite, and the data was normalized and filled with missing values. The obtained data matrix was subjected to subsequent bioinformatic difference test and statistical analysis, including 3 statistical methods of random forest, PLS-DA and LASSO regression analysis, to screen out a ranking list of the most effective differential metabolites between samples of patients with acute myocardial infarction and control samples. As shown in FIG. 1, 30 differential metabolites were screened out through each of the 3 statistical methods of random forest, PLS-DA, and LASSO regression analysis. Finally, the metabolites screened out in all of the 3 methods were further selected as biomarkers for predicting early myocardial infarction, as shown in Table 1.

TABLE 1
13 biomarkers for predicting myocardial infarction

| | | | SUPER | |
|---|---|---|---|---|
| Serial | | | META | |
| number | English name | CAS ID | PATHWAY | SUB META PATHWAY |
| 1 | pentadecanoate | 1002-84-2 | Lipid | Long Chain Saturated |
| | (15:0) | | | Fatty Acid |
| 2 | tryptophan | 73-22-3 | Amino Acid | Tryptophan Metabolism |
| 3 | laurate (12:0) | 143-07-7 | Lipid | Medium Chain Fatty Acid |
| 4 | methionine | 3226-65-1 | Amino Acid | Methionine, Cysteine, |
| | sulfoxide | | | SAM and Taurine |
| | | | | Metabolism |
| 5 | (14 or 15)- | 112-39-0 | Lipid | Fatty Acid, Branched |
| | methylpalmitate | | | |
| | (a17:0 or i17:0) | | | |
| 6 | acetylcarnitine (C2) | 3040-38-8 | Lipid | Fatty Acid Metabolism |
| | | | | (Acyl Carnitine, Short |
| | | | | Chain) |
| 7 | 3-methyl-2- | 759-05-7 | Amino Acid | Leucine, Isoleucine and |
| | oxobutyrate | | | Valine Metabolism |
| 8 | oxindolylalanine | 32999-55-6 | Amino Acid | Tryptophan Metabolism |
| 9 | asparagine | 70-47-3 | Amino Acid | Alanine and Aspartate |
| | | | | Metabolism |
| 10 | methionine | 63-68-3 | Amino Acid | Methionine, Cysteine, |
| | | | | SAM and Taurine |
| | | | | Metabolism |
| 11 | N-palmitoylglycine | 158305-64-7 | Lipid | Fatty Acid Metabolism |
| | | | | (Acyl Glycine) |
| 12 | carnitine | 541-15-1 | Lipid | Carnitine Metabolism |
| 13 | phenylalanine | 63-91-2 | Amino Acid | Phenylalanine Metabolism |

The 13 metabolites screened out in this example were significantly different between the plasma of patients with early acute myocardial infarction and of normal people, and could effectively distinguish the patients with early acute myocardial infarction from the normal people.

### Example 2 Comparative Analysis of Performance Using Different Markers and a Combination Thereof

### 1. Individual Biomarker

This example analyzed the individual diagnostic performance of the 13 biomarkers obtained in Example 1 that could distinguish 86 patients with early acute myocardial infarction and 114 normal people, respectively, and calculated their AUC values, thresholds, specificity and sensitivity, and the results were shown in Table 2.

TABLE 2
Performance of a single marker in diagnosing
early myocardial infarction

| Serial | | | Thresh- | Speci- | Sensi- |
|---|---|---|---|---|---|
| number | Biomarker | AUC | old | ficity | tivity |
| 1 | pentadecanoate | 0.940 | 25.615 | 0.964 | 0.793 |
| | (15:0) | | | | |
| 2 | tryptophan | 0.926 | 28.636 | 0.893 | 0.828 |
| 3 | laurate (12:0) | 0.947 | 26.719 | 0.857 | 0.897 |
| 4 | methionine | 0.954 | 22.775 | 0.893 | 0.897 |
| | sulfoxide | | | | |
| 5 | (14 or 15)- | 0.922 | 23.898 | 0.893 | 0.862 |
| | methylpalmitate | | | | |
| | (a17:0 or i17:0) | | | | |
| 6 | acetylcarnitine (C2) | 0.909 | 28.014 | 0.964 | 0.724 |
| 7 | 3-methyl-2- | 0.866 | 24.544 | 0.75 | 0.897 |
| | oxobutyrate | | | | |
| 8 | oxindolylalanine | 0.905 | 19.921 | 0.893 | 0.862 |
| 9 | asparagine | 0.877 | 27.23 | 0.929 | 0.655 |
| 10 | methionine | 0.871 | 27.717 | 0.821 | 0.862 |
| 11 | N-palmitoylglycine | 0.856 | 19.322 | 0.714 | 0.931 |
| 12 | carnitine | 0.845 | 29.411 | 0.893 | 0.724 |
| 13 | phenylalanine | 0.825 | 29.338 | 0.893 | 0.69 |

It could be seen from Table 2 that the AUC values of the 13 biomarkers provided in Example 1 for diagnosing early myocardial infarction were all high, all of which could reach above 0.825. The ROC analysis chart of asparagine as shown in FIG. 2 had high accuracy, and was a brand-newly discovered marker that could be used for screening early myocardial infarction efficiently, indicating that these 13 biomarkers could perform diagnosis of early myocardial infarction individually.

### 2. Combination of Multiple Biomarkers

Although the aforementioned individual biomarkers could also distinguish the patients with early myocardial infarction from normal people, generally speaking, multiple biomarkers were combined to further improve the accuracy, specificity and sensitivity of prediction.

However, when combined with one or more other biomarkers, an individual biomarker with higher accuracy in early diagnosis of acute myocardial infarction might not necessarily play a greater role in the combination, and meanwhile it is not a biomarker. The greater the number, it was not that the more the number of biomarkers, the higher the prediction accuracy (AUC value) of their combination, so a large number of verification experiments were needed.

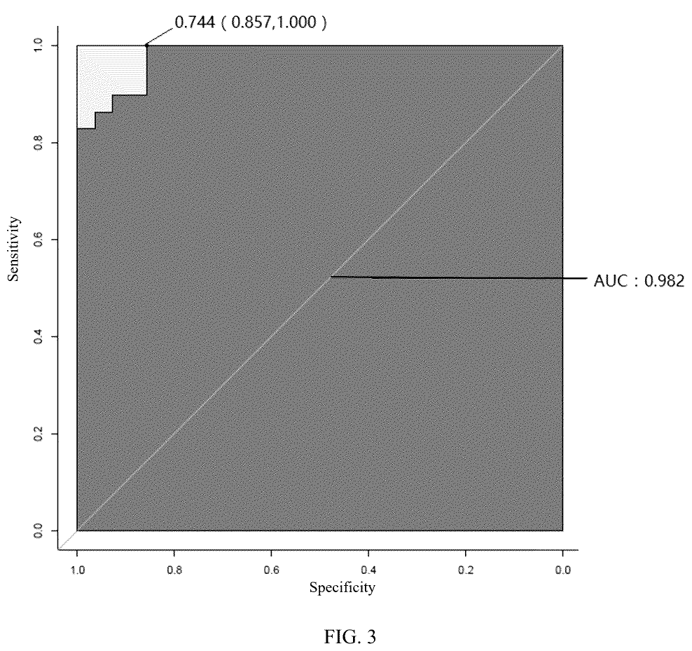

This example adopted the 13 biomarkers provided in Example 1 and adopted logistic regression analysis to construct a diagnostic model for early myocardial infarction. Blood samples of 57 cases (known to include 28 positive myocardial infarction patients and 29 negative normal people) were subjected to predictive analysis, and the prediction results were shown in FIG. 3.

It could be seen from FIG. 3 that adopting the logistic regression analysis model could obtain better prediction results of early myocardial infarction, and truly realize the noninvasive and global screening of early myocardial infarction with higher sensitivity and specificity, which fully met the clinical needs.

### Example 3 Development and Verification of a Probability Model for Predicting Early Myocardial Infarction

This example combined the 13 metabolites screened out in Example 2, and used logistic regression to establish a probability model for predicting the occurrence of early myocardial infarction. It was hoped that a model could be established for the metabolites and had ideal predictive performance.

The model formula was:

$$log ( \frac{p}{1 - p} ) = - 1.27416652 {9}^{*} + pentadecanoate + 1.078442206 * tryptophan - 1.27535689 * laurate + 1.32331323 * methionine sulfoxide - 1.010616893 * methylpalmitate - 1.450150532 * acetylcarnitine - 0.720980628 * 3 - methyl - 2 - oxobutyrate + 0.732863088 * oxindolylalanine + 0.494666685 * asparigine + 1.114785457 * methionine - 0.989479547 * N - palmitoylglycine + 2.207517356 * carnitine + 1.378071365 * phenylalanine ;$$

In the discovery set, the AUC of the model established with the 13 metabolites in predicting the probability of early myocardial infarction was 0.982, proving that the predictive power of a combination of the biomarkers was higher than that of individual metabolite indicators.

Studies had shown that for the model established with the 13 metabolites, if the predictive value p was greater than 0.53, the predicted probability and risk of early myocardial infarction was high; and if p was less than 0.53, the predicted probability and risk of early myocardial infarction was low.

All patents and publications mentioned in the specification of the present invention indicate that they are published techniques in the art and can be used by the present invention. All patents and publications cited herein are also listed in the references as if each publication is specifically and individually cited. The present invention described herein may be practiced in the absence of any element or elements, limitation or limitations, and here such a limitation is not specifically stated. For example, in each instance here the terms “comprising/including”, “consisting essentially of” and “consisting of” may be replaced by one of the remaining 2 terms. The so-called “a/an” here only means “one”, and it does not exclude that it only includes one, or alternatively it can also mean including 2 or more. The terms and expressions adopted here are for description rather than limiting, and there is no intention to indicate that these terms and explanations described in this specification exclude any equivalent features, but it can be known that any suitable changes or modifications can be made within the scope of the present invention and the claims. It can be understood that the examples described in the present invention are some preferred examples and features. Some modifications and changes can be made by any person of ordinary skills in the art based on the essence of the description of the present invention, and these modifications and changes are also considered to belong to the scope of the present invention and the scope limited by the independent claims and the dependent claims.

1. A method of predicting whether an individual is suffering from acute myocardial infarction, comprising:
providing a liquid sample derived from an individual;
detecting a concentration or a amount of a biomarker in the liquid sample to obtain a detection value; and
determining whether the individual is suffering from acute myocardial infarction based on the detection value,
wherein the biomarker comprises a combination of the following markers:
pentadecanoate, tryptophan, laurate, methionine sulfoxide, methylpalmitate, acetylcarnitine, 3-methyl-2-oxobutyrate, oxindolylalanine, asparagine, methionine, N-palmitoylglycine, carnitine, and phenylalanine.

2. The method according to claim 1, wherein the acute myocardial infarction comprises a hyperacute phase, and the hyperacute phase refers to a time window range of a patient after the acute myocardial infarction occurs.

3. The method according to claim 2, wherein the acute myocardial infarction comprises a hyperacute phase, and the hyperacute phase means that the patient will progress to acute myocardial infarction within 6 hours or less than 6 hours.

4. The method according to claim 3, wherein the liquid sample comprises blood, urine, saliva or sweat.

5. The method according to claim 4, wherein the blood sample is a plasma sample.

6. The method according to claim 4, wherein the method further comprises: providing a system for diagnosing whether an individual is in the hyperacute phase of acute myocardial infarction, wherein the system comprises a data analysis module for analyzing the detection value of the biomarker combination in the blood sample.

7. The method according to claim 8, wherein the data analysis module calculates a predicted value of predicting that the patient is in the hyperacute phase of acute myocardial infarction by substituting the detected value of each marker in the biomarker combination into a logistic regression equation, so as to judge whether the patient will progress to acute myocardial infarction within 6-8 hours, and the logistic regression equation is:
$$log ( \frac{p}{1 - p} ) = - 1.27416652 {9}^{*} + pentadecanoate + 1.078442206 * tryptophan - 1.27535689 * laurate + 1.32331323 * methionine sulfoxide - 1.010616893 * methylpalmitate - 1.450150532 * acetylcarnitine - 0.720980628 * 3 - methyl - 2 - oxobutyrate + 0.732863088 * oxindolylalanine + 0.494666685 * asparigine + 1.114785457 * methionine - 0.989479547 * N - palmitoylglycine + 2.207517356 * carnitine + 1.378071365 * phenylalanine ;$$
wherein p represents a critical threshold when the patient is in the hyperacute phase of acute myocardial infarction.

8. The method according to claim 7, wherein the risk of the patient being in the hyperacute phase of acute myocardial infarction is predicted to be high when p is greater than 0.53; and the risk of the patient being in the hyperacute phase of acute myocardial infarction is predicted to be low when p is less than 0.53.

9. The method according to claim 8, wherein the system further comprises a data storage module, a data input interface and a data output interface, the data storage module is used for storing the detection value of a biomarker, the data input interface is used for inputting the detection value of the biomarker, and the data output interface is used for outputting a prediction result.

10. The method according to claim 7, wherein the detection value comprises a detection value obtained by detecting the marker combination using ultra-high performance liquid chromatography-tandem mass spectrometry UPLC-MS/MS.
